(19) United States
(12) Patent Application Publication
(10) Pub. No.: US 2014/0275235 A1
(43) Pub. Date: Sep. 18, 2014

(54) Treatment of Proliferative Disorders

(71) Applicants: Loic Pierre Deleyrolle, Gainesville, FL (US); Brent Allan Reynolds, Gainesville, FL (US)

(72) Inventors: Loic Pierre Deleyrolle, Gainesville, FL (US); Brent Allan Reynolds, Gainesville, FL (US)

(21) Appl. No.: 14/197,897

(22) Filed: Mar. 5, 2014

Related U.S. Application Data
(60) Provisional application No. 61/791,539, filed on Mar. 15, 2013.

(51) Int. Cl. A23L 1/29; A61K 36/31; A61K 31/12; A61K 31/353

(52) U.S. Cl. CPC A23L 1/293; A61K 31/353; A61K 36/31; A61K 31/12
USPC 514/456

(57) ABSTRACT

The present invention relates to a method and composition for treating safely (in a non-toxic way) disease or disorder exhibiting excessive cellular proliferation comprising administering to a patient needing such treatment, a composition comprising curcumin (derived from turmeric), epigallocatechin-3-gallate (EGCG, enriched in green tea), glucosinolates (enriched in cruciferous vegetables) and/or derivatives thereof, such as sulforaphane (SFN), alone or combined with a ketogenic diet or a modified ketogenic diet. Also the current invention relates to a composition comprising medium chain triglycerides, Epigallocatechin-3-gallate, curcumin, compositions comprising glucosinolates and/or derivatives thereof, such as sulforaphane (SFN). The invention provides that administering a composition comprising curcumin, EGCG, glucosinolates, alone or combined with a ketogenic diet or a modified ketogenic diet targets malignant cells leading to decrease cellular proliferation and increase survival of the subject treated with the current invention.

### BACKGROUND OF THE INVENTION

Due to a shift in most cancer cells from oxidative phosphorylation to aerobic glycolysis (known as the Warburg effect) cancer can be viewed as a metabolic disease where energy flux is shifted from a highly efficient method of generating energy (36 molecules of ATP from 1 molecule of glucose) to an inefficient method (4 molecules of ATP from one molecule of glucose). The result is that cancer cells expend an enormous amount of glucose to survive and multiply. While controversy exists as to the relationship between the Warburg effect and altered signaling pathways, the combined reliance of tumor cells for excessive amounts of glucose and signaling pathway alterations suggest that targeting these two related phenomena may provide better outcomes in cancer treatment.

Most cancer treatments employ the use of toxic chemicals aimed at killing cancerous cells. While these treatments are highly effective, unfortunately, they have similar effects on normal, non-cancerous cells as well. The key to developing an effective and well-tolerated chemotherapy regime is to balance the positive tumor killing effects of the compounds with the toxic side effects. The use of non-toxic compounds that are able to target altered signaling pathways and influence energy flux may provide an effective and tolerable treatment. An additional advantage of using non-toxic approach is the ability to apply multiple agents simultaneously with reduced chances of cumulative toxicity. The current invention provides treatment of proliferative disorders that target altered signaling pathways and energy flux in cancerous cells.

### BRIEF SUMMARY OF THE INVENTION

The disclosed invention is a treatment for diseases that are defined by excessive cellular proliferation, such as cancer. The invention provides a composition of several nutraceuticals and/or processed food products that may be used together with a dietary regime that is intended to mimic the physiological effects of a ketogenic diet [KD]. Hence, the invention provides a treatment for a patient with a disease or disorder where excessive cellular proliferation is paramount, such as cancer. The treatment involves administering to the patient a KD or a modified ketogenic diet [mKD] in addition to consuming one or more of the following nutraceuticals; epigallocatechin-3-gallate (EGCG), curcumin, and compositions comprising glucosinolates and/or its derivatives [such as glucoraphanin and/or suiforaphane (SFN), as can be found in broccoli sprouts or sprouts of other cruciferous vegetables or in cruciferous vegetables themselves]. The mKD is achieved by having the patient consume a low carbohydrate diet which is supplemented with medium chain triglycerides [MCT]. The application of EGCG, curcumin, and compositions comprising glucosinolates and/or its derivatives either individually and/or in combination with the mKD or KD is able to control, manage, inhibit or reverse excessive cellular proliferation, such as that seen in, but not solely including, cancer.

### BRIEF DESCRIPTION OF DRAWINGS

FIG. 1: mKD/NUT treatment decreases circulating glucose level The disclosed invention relates to two components, i) a dietary intervention (KD or mKD) aiming to influence the energy flux in malignant cells via reproduction of the physiological effects of a ketogenic diet (i.e. lower glucose level and increase ketones concentration) and ii) a combination of nutraceuticals (NUT) able to target multiple signaling pathways driving tumor cell proliferation.
Blood glucose level was compared between animals that were fed for 2 weeks with one of the two different diets [control or mKD/NUT]. Glucose levels were significantly decreased in the mKD/NUT fed animals compared to control. [**, p<0.005, t-test]. This data demonstrates that or the mKD/NUT treatment is able to reproduce the KD physiological effect of significantly decreasing blood glucose level.
Treatments composition is as follow:
[1] Control=55% carbohydrates, 30% proteins, 15% fats
[2] mKD/NUT=mKD (20% carbohydrates, 50% fats (about half coming from MCT, Neobee 598), 30% Proteins)+Nutraceuticals (NUT=Broccoli Sprout Powder (BSP) [20 g/kg of body weight], Curcumin [1200 mg/kg of body weight], EGCG [1200 mg/kg of body weight]).

FIG. 2: mKD/NUT treatment increases the level of circulating ketone bodies
Blood ketones level was compared between animals that were fed respectively for 2 weeks with the different diets [control, mKD/NUT]. Ketones level was significantly decreased in the mKD/NUT fed animals compared to control. **, p<0.005, t-test. This data demonstrates that our treatment mKD/NUT is able to reproduce the KD physiological effect of increasing significantly blood ketones level.
Treatments composition is as follow:
[1] Control=55% carbohydrates, 30% proteins, 15% fats
[2] mKD/NUT=mKD (20% carbohydrates, 50% fats (about half coming from MOT, Neobee 598), 30% Proteins)+Nutraceuticals (NUT=Broccoli Sprout Powder (BSP) [20 g/kg of body weight], Curcumin [1200 mg/kg of body weight], EGCG [1200 mg/kg of body weight]).

FIG. 3: General safety of mKD/NUT—effect on body weight
Toxicity of mKD/NUT was assessed by monitoring body weight over 16 days. No difference was observed in the body weight evolution between the control and mKD/NUT groups (p=0.111, 2 way ANOVA). These results teach that the treatment mKD/NUT represents a safe, non-toxic therapeutic intervention.
Treatments composition is as follow:
[1] Control=55% carbohydrates, 30% proteins, 15% fats
[2] mKD/NUT=mKD (20% carbohydrates, 50% fats (about half coming from MCT, Neobee 598), 30% Proteins)+Nutraceuticals (NUT=Broccoli Sprout Powder (BSP) [20 g/kg of body weight], Curcumin [1200 mg/kg of body weight], EGCG [1200 mg/kg of body weight]).

FIG. 4: Effect of mKD/NUT on Progression Free Survival NOD/SCID animals were implanted subcutaneously with 1 million human glioblastoma (hGB) cells in the right flank. Tumor progression was followed using calipers by recording 2 measurements of tumor diameter and converting this into a volume using the following formula: (4/3)ΠR₃. For spheroid tumors the two measurements were averaged to determine the diameter of the sphere. In the case of ellipsoid tumors (Le. prolate or oblate spheroid mass) the formula used was: (4/3)Π*(d/2)*(d/2)₂. In this case the second measurement “d₂” would count twice and “d” only once. For prolate spheroids, the long measurement occurs once while the short measurement occurs twice. Conversely, for the oblate spheroid tumors, the long measurement occurs twice while the short one occurs only once. Following this criteria, tumor volume was calculated 3×/week and the time for a tumor to reach a significant size [300 mm₃] was calculated. Treatments were initiated when a palpable mass was identified [approximately 65 mm₃]. Animals treated with mKD/NUT demonstrated a significantly greater progression free survival (time during which tumor volume is maintained lower than 300 mm₃) compared to controls (40% improvement). ***, p<0.0001, West). This data indicates that our unique combination of a modified ketogenic diet (mKD) with a mixture of bioactive nutraceuticals (derived from green tea extract, turmeric and calciferous vegetables powder such as broccoli sprout powder) represents an efficient treatment to delay tumor progression.
Treatments composition is as follow:
[1]

Control=55% carbohydrates, 30% proteins, 15% fats
[2] mKD/NUT=mKD (20% carbohydrates, 50% fats (about half coming from MCT, Neobee 598), 30% Proteins)+Nutraceuticals (NUT=Broccoli Sprout Powder (BSP) [20 g/kg of body weight], Curcumin [1200 mg/kg of body weight], EGCG [1200 mg/ka of body weight]).

FIG. 5: Effect of mKD/NUT on Overall survival
NOD/SCID animals were implanted subcutaneously with 1 million hGB cells in the right flank. Tumor dimensions were monitored 3×/week using calipers and volume was calculated. Treatments were initiated when a palpable mass was identified [approximately 65 mm₃]. Animals were sacrificed when they reached endpoint (1700 mm₃). The average time to reach endpoint volume was then compared. Animals treated with mKD/NUT showed a significant improvement (approximately 25%) over controls (**, p<0.005, West). This data indicates that our unique combination of a modified ketogenic diet (mKD) with a mixture of bioactive nutraceuticals (derived from green tea extract, turmeric and calciferous vegetables powder such as broccoli sprout powder) is able to increase the mean time of survival in animal model of cancer.

### DETAILED DESCRIPTION OF THE INVENTION

Nutraceutical Supplementation: Nutraceutical [NUT] is composed of 3 nutraceuticals that have demonstrated anticancer properties, are non-toxic and individually have a documented safety profile. The nutraceuticals are: [1] curcumin, [2] glucosinolates and derivatives such as sulforaphane, [derived from broccoli sprout powder—BSP] and [3] a green tea catechin, epigallocatechin 3-gallate [EGCG]. Each of these agents has demonstrated efficacy in regulating tumor cell proliferation and progression using multiple mechanisms ranging from increasing apoptosis, regulating cell cycle genes, down regulating oncogenic pathways and altering epigenetic regulation of gene expression^{1,2 3 4 5}. Each nutraceutical targets multiple mechanisms that influence tumor cell behavior and overlap exists such that two or more of the biologically active compounds targets individual pathways. This allows us to apply established ecological principles of pest control to cancer therapy. In general, the approach focuses on the use of non-toxic reagents to manage a pest population by saturating the ecosystem so the drivers of population growth are targeted at multiple levels⁶. This reduces the efficiency of the population to successfully develop and execute an escape strategy. In a more cell/cancer biology perspective, this can also be viewed as implementing horizontal (multiple pathways) and vertical (several steps in a given pathway) targeting of critical pathways. This is relevant given the multiple molecular mechanisms involved in supporting tumor cell proliferation and the crosstalk that occurs between the different mechanisms responsible for growth and the development of resistance. As a result of this heterogeneity, signaling pathway crosstalk and adaptation, single-target inhibition may be insufficient to elicit a sustainable antitumor effect. A combination of several therapeutic compounds would be preferred. While the application of multiple agents in cancer therapy is not new, the approach is often limited by cumulative toxicity when several drugs are delivered simultaneously. However, with our natural product approach this does not occur as each compound has a well documented safety profile [both preclinical and clinical] when delivered at doses that are effective at altering the drivers and contributors to tumor cell survival, proliferation and development of resistance.

EGCG: Epigallocatechin-3-gallate is the most abundant catechin in green tea, which is the most consumed beverage worldwide after water. Polyphenols derived from green tea are well-known antioxidants, have demonstrated inhibit tumor cell proliferation in multiple animal models of cancer. These effects are due to the ability of EGCG to decrease cell proliferation, increase apoptosis, suppress angiogenesis and affect a number of molecular pathways that contribute to the development of resistance and cancer robustness. These actions are seen at micromolar concentrations that can be achieved by oral ingestion of ECGC².

Curcumin: Curcumin is the active component of the dietary spice turmeric, the yellow pigment in curry powder and has been used in traditional medicine for the treatment of inflammation and multiple diseases. The biological functions of curcumin are diverse and range from anti-tumor, anti-oxidative, anti-viral, anti-amyloid, anti-bacterial and anti-hepatotoxic activities⁷. In the past 20 years hundreds of papers have been published investigating the underlying mechanisms and they appear to occur via regulation of a number of molecular targets⁸. Data indicates that curcumin has a diverse range of molecular targets, and hence is a promising agent to be used in combination with other molecules to vertically and horizontally inhibit pathways driving tumor proliferation and robustness. Curcumin is known to physically bind to 33 different proteins and to modulate molecular targets including transcription factors, growth factors and their receptors, kinases and enzymes known to play a role in proliferation, invasion, metastasis, angiogenesis and resistance to chemotherapy. Some of the molecular targets in cancer cells include: [1] NF-kB, [2] AP-1, [3] STAT-3, [4] HIF-1, [5] EGFR and [6] Notch-1⁸.

Cruciferous Vegetable Sprout Powder: Many of the anticancer effects of cruciferous vegetables have been attributed to isothiocyanates [ITC] that are formed by hydrolysis of their precursor parent molecule glucosinolates. One of the most studied cruciferous vegetable ITCs is sulforaphane [SFN] whose precursor glucoraphanin [GRP] is abundant in broccoli, cauliflower and cabbage, with its highest concentration being found in broccoli sprouts. Hydrolysis of GRP requires the activity of myrosinase which is present in the vegetables themselves and in microflora of the colon⁹. The most efficient conversion occurs following mechanical breakdown of the plant cell wall [i.e. chewing] and release of myrosinase which converts GRP into SFN and also gives foods such as broccoli a particular bitter or pungent odor and taste¹⁰. SFN is rapidly absorbed with 80% bioavailability, attains peak plasma levels within 2 hours and is characterized by a long terminal elimination phase^{11,12}. A great deal of research has gone into studying SFN's ability to simultaneously modulate multiple cellular targets related to cancer development. These include its ability to protect DNA by altering carcinogen-metabolizing enzymes and blocking mutagens, inhibiting proliferation, inducing apoptosis, inhibiting angiogenesis and inhibiting histone deacetylase¹³. SFN has been shown to inhibit malignant progression of lung adenomas¹⁴ and to selectively target benign hyperplasia cells, and cancerous cells, while leaving normal prostate cells unaffected¹⁵. The combination of SFN together with traditional chemotherapy compounds such as cisplatin and doxorubicin has demonstrated increased cancer cell killing in both pancreatic and prostate cancer and targeting of cancer stem cells¹⁶. A 12-month study demonstrated that a broccoli rich diet altered androgen signaling and regulated EGF, TGF-beta 1 and insulin signaling in men with high-grade prostatic intraepithelial neoplasia¹⁷. In addition, SFN is a potent HDAC inhibitor^{13, 18, 19}.

In summary, the compounds contained within cruciferous vegetables and powders and concentrates extracts obtained from them, targets a multitude of cancer promoting mechanisms that can be effectively regulated with physiological concentrations of SFN obtained through dietary intervention.

Combining Therapeutics to Achieve Horizontal and Vertical Inhibition: Central to our approach is the application of multiple therapeutic agents that target a variety of pathways and mechanisms responsible for uncontrolled proliferation. Due to the heterogeneous nature of most solid tissue cancers, targeting single pathways or mechanisms allows the selection pressure to evoke escape strategies at both the cell and population level. The result is the development of resistance through selection and dependence on alternative mechanisms to support continuous cellular growth. A solution is the application of multiple agents that target more than one pathway, however this can be complicated by additive toxicity when more than one chemo- or biological-therapy is used. A strategy to address this challenge is to use individual agents that have low toxicity and a broad safety profile. Many natural products fit this description. As described above, each of our natural products (nutraceuticals) targets multiple signaling pathways that regulate tumor cell survival and proliferation. When combined together a variety of independent mechanisms that support tumor growth can be targeted [horizontal inhibition] and even individual pathways can be targeted at multiple points [vertical inhibition]. The result is a continued selection pressure placed on the tumor population that dampens or delays the emergence of resistance. Hence, NUT is a combinatorial anti proliferation treatment involving the use of 3 nutraceutical products delivered as a dietary supplement. This experimental therapeutic has demonstrated efficacy in altering tumor cell proliferation, tumor progression and extending lifespan with no reported toxicity.

Ketogenic and modified Ketogenic Diet: In the 1920's it was observed that fasting was found to be beneficial to patients with difficult to control seizures and hence the ketogenic diet [KD] was implemented as an anti-epileptic treatment mimicking the biochemical changes associated with starvation. The KD is a high-fat, low-protein, low-carbohydrate diet that depletes the patients carbohydrate and glycogen stores to cause a shift in the body's metabolism toward a state of starvation. When glucose levels are scarce in the circulating system, the body's compensatory mechanism produces energy from the liver. Activation of hormone-sensitive lipases from adipose tissue mobilizes non-esterified fatty acids (NEFA), which provide substrates for the hepatic production of ketone bodies. In contrast to normal brain cells, which evolved to metabolize ketone bodies for energy when glucose levels are reduced, most brain tumor cells are dependent on glycolysis for survival and are unable to metabolize ketone bodies for energy. Multiple researchers have demonstrated that the use of a ketogenic diet causes a reduction in blood glucose, an elevation in blood ketones and extends life in mouse models of malignant cancers. Armed with this knowledge and with the resurgence of tumor metabolism in the clinic, doctors were testing the efficacy of the ketogenic diet on cancer patients. Sparingly, case reports have been published showing the effectiveness of the ketogenic diet on a case-by-case basis. Their findings were similar in that the ketogenic therapies, which lower blood glucose levels while elevating ketone body levels, could be an effective non-toxic therapy for increasing progression free survival in patients with malignant brain tumor.

Medium-chain triglycerides (MCTs): Fat from the ketogenic diet is specific in its ability to increase circulating ketone levels. Long and very-long chain fatty acids require modification in the hepatic portal prior to being released into the bloodstream. Medium and small-chain fatty acids have the ability to bypass this hepatic portal and move ketones straight into the bloodstream, increasing ketone levels when ingested on their own, even in the presence of carbohydrates. Medium-chain triglycerides (MCTs) are a commercially available product that can be used to increase circulating ketone levels.

Increased ketone and reduced glucose concentrations are the primary physiological effects of a ketogenic diet (for example, a diet composed of 90% fat and 10% proteins/carbohydrates)

The modified ketogenic diet [mKD] is intended to mimic the ketogenic diet without the extreme carbohydrate restriction [5%]. While carbohydrates are reduced [10-20%] from that found in a normal diet [50%] combining this with supplementation of MCTs, one is able to replicate the key primary physiological features of the KD, reduced glucose and increased ketones. Hence, the mKD, relative to total caloric intake can contain approximately 5% to about 20% carbohydrates, 30% to 75% fats and 5% to 65% proteins. In certain embodiments MCT may make up about 50% of the fat content of the subject's diet.

The ketogenic diet (KD) is a diet wherein the carbohydrate content is less than, or equal to, about 5% of the total caloric intake the subject each day and the balance of the diet consists of fats or proteins. Thus, the diet provides, as a function of total caloric intake each day, about 5% or less carbohydrate, about 30% to about 90% fat and about 5% to about 70% protein. In certain embodiments, the diet provides about 3% (or less) carbohydrate, about 57% to about 95% fat, about 5% to about 40% protein. In some embodiments, from about 30% to about 70% (e.g., about 30%, about 40%, about 50%, about 60% or about 70%) of the fat content of the subject's diet can be made up of medium chain triglycerides (MCT). Other embodiments provide that MCT make up about 50% of the fat content of the subject's diet.

### EXAMPLES

The treatment mKD/NUT (combining a modified Ketogenic Diet [mKD] with a mixture of Nutraceuticals [NUT]) alters glucose and ketone levels.
NOD-SCID animals were placed on a mKD+NUT for two weeks. The composition of the diets are as follows (expressed as percentage of calories):
Control Diet: Is a standard mouse chow and is composed of 55% carbohydrates, 30% proteins, 15% fats
mKD/NUT: mKD (20% carbohydrates, 50% fats (about half corning from MCT, Neobee 598), 30% Proteins)+Nutraceuticals (NUT=BSP [20 g/kg of body weight], Curcumin [1200 mg/kg of body weight], EGCG [1200 mg/kg of body weight]).
Fifteen and thirteen animals (control and mKD/NUT group respectively), were placed on their respective diet regimen for 2 weeks at which point tail tip method was used to collect blood via the following protocol: “Using a 50 mL conical (or mouse restrained place a non-anesthetized mouse gently inside grasping the mouse by the tail. Place the mouse's tail on a hard surface. Using a scalpel cut off less than 1 mm of the tip of the tall. Place your fingers at the base of the tail and gently squeeze upward running your fingers from base to tip of the tail. One to two drops of blood (5-10 μL) will appear at the tip of the tail, Using a thy gauze wipe away first few drops of blood and repeat steps until desired amount of blood is collected (should be dark, whole blood, clear [plasma like] blood will give you an inconsistent blood reading). Place the mouse back into the cage and monitor for excess bleeding”.
Blood samples were analyzed with Precision Xtra blood glucose/ketone monitor and expressed in mg/dl and mmol for glucose and ketones, respectively. With regards to glucose levels, the mKD/NUT diet resulted in a significant reduction in glucose [FIG. 1, p<0.005]. Conversely, ketone levels were elevated in the dietary group mKD/NUT [FIG. 2, p<0.005]. These data demonstrate that the mKD/NUT diet is able to mimic the two key physiological features of a ketogenic diet, a significant reduction in glucose and a significant increase in ketones.

The mKD/NUT diet is safe and has no sign of toxicity.
The vast majority of cancer therapeutics have dose-limiting toxic side effects that impact not only a patient's well being but also result in suspended treatments and reduced dosages that can impact on treatment efficacy. One of the best indicators of overall health in a rodent is its body weight [this is true in human patients as well]. NOD-SCID animals were implanted subcutaneously with 1 million of human glioblastoma cell (hGB) cells [patient derived lines] into the right flank. Animals were monitored 3×/week for signs of tumor formation. Once a tumor was identified [by palpation & approximately 65 mm3] animals were randomly assigned to 1 or 2 groups: [1] Control diet or [2] mKD/NUT diet. Body weight was monitored 14 times over the course of the experiment. FIG. 3 depicts percentage change in body weight till the first control animal reached endpoint [16 days]. While the mKD/NUT group initially lost weight over the first few days [due to adjusting to a new diet], they quickly recovered the lost weight and continued to gain weight similarly than control [p=0.111, 2 way ANOVA]. These results support the conclusion that mKD/NUT diet is safe, is nutritionally sufficient to support normal health. and has no noted toxic side effects.

The mKD/NUT diet represents an effective anticancer treatment.
NOD-SCID animals were placed on one of the following diets:
[1] Control diet: Is a standard mouse chow and is composed of 55% carbohydrate, 30% protein, 15% fat.
[2] mKD/NUT: mKD (20% carbohydrates, 50% fats (about half coming from MCT. Neobee 598), 30% Proteins) +Nutraceuticals (NUT=BSP [20 g/kg of body weight], Curcumin [1200 mg/kg of body weight], EGCG [1200 mg/kg of body weight]).
Following subcutaneous implant of 1 million of hGB tumor cells into the right flank of NOD-SCID mice, animals were randomized and put on one of the two diets. Tumor progression was followed by recording 2 measurements of tumor diameter and converting this into a volume using the following formula: (4/3)ΠR₃. For spheroid tumors the two measurements were averaged to determine the diameter of the sphere. In the case of ellipsoid tumors (i.e. prolate or oblate spheroid mass) the formula used was: (4/3)Yr*(d/2)*(d/2)₂. In this case the second measurement “d₂” would count twice and “d” only once. For prolate spheroids, the long measurement occurs once while the short measurement occurs twice. Conversely, for the oblate spheroid tumors, the long measurement occurs twice while the short one occurs only once. Following this criteria, tumor volume was tracked 3×/week and the time from a barely palpable tumor [approximately 65 mm₃] to a tumor of a significant size [300 mm₃] was calculated, defining the time during which the tumor is considered “not progressing” determining the progression free survival period. FIG. 4 illustrates that mKD/NUT treatment results in a significant increase in progression free survival. The average time to reach endpoint (defined by a tumor volume greater than 1700 mm³) was also compared [FIG. 5]. Similar to time to progression, mKD/NUT increased significantly the mean survival.
Overall, these data support our conclusion that our unique treatment mKD/NUT is nutritionally sufficient with no adverse effects on overall health and is an effective cancer treatment that delays tumor progression and enhances overall survival.

### REFERENCES

1 Khan, Afaq, Saleem, Ahmad & Mukhtar. Targeting multiple signaling pathways by green tea polyphenol (-)-epigallocatechin-3-gallate. Cancer Res 66, 2500-2505, doi:10.1158/0008-5472.CAN-05-3636 (2006).
2 Yang, C., Wang, X., Lu, G. & Picinich, S. Cancer prevention by tea: animal studies, molecular mechanisms and human relevance. Nat Rev Cancer, doi:10.1038/nrc2641 (2009).
3 Teiten, M.-H., Eifes, S., Dicato, M. & Diederich, M. Curcumin—The Paradigm of a Multi-Target Natural Compound with Applications in Cancer Prevention and Treatment. Toxins 2, 128-161, doi:10.3390/toxins2010128 (2010).
4 Clarke, J. D., Dashwood, R. H. & Ho, E. Multi-targeted prevention of cancer by sulforaphane. Cancer letters 269, 291-304, doi:10.1016/j.canlet.2008.04.018 (2008).
5 Fimognari, C. Sulforaphane as a promising molecule for fighting cancer. Mutation Research/Reviews in Mutation Research (2007).
6 Kogan, M. Integrated pest management: historical perspectives and contemporary developments. Annual review of entomology 43, 243-270, doi:10.1146/annurev.ento.43.1.243 (1998).
7 Maheshwari, R. K., Singh, A. K., Gaddipati, J. & Srimal, R. C. Multiple biological activities of curcumin: a short review. Life Sciences 78, 2081-2087, doi:10.1016/j.lfs.2005.12.007 (2006).
8 Kunnumakkara, A. B., Anand, P. & Aggarwal, B. B. Curcumin inhibits proliferation, invasion, angiogenesis and metastasis of different cancers through interaction with multiple cell signaling proteins. Cancer letters 269, 199-225, doi:10.1016/j.canlet.2008.03.009 (2008).
9 Getahun, S. M. & Chung, F. L. Conversion of glucosinolates to isothiocyanates in humans after ingestion of cooked watercress. Cancer epidemiology, biomarkers &amp; prevention: a publication of the American Association for Cancer Research, cosponsored by the American Society of Preventive Oncology 8, 447-451 (1999).
10 Shapiro, T. A. T., Fahey, J. W. J., Wade, K. L. K., Stephenson, K. K. K. & Talalay, P. P. Chemoprotective glucosinolates and isothiocyanates of broccoli sprouts: metabolism and excretion in humans. Cancer epidemiology, biomarkers &amp; prevention : a publication of the American Association for Cancer Research, cosponsored by the American Society of Preventive Oncology 10, 501-508 (2001).
11 Hanlon, N. et al. Absolute bioavailability and dose-dependent pharmacokinetic behaviour of dietary doses of the chemopreventive isothiocyanate sulforaphane in rat. British Journal of Nutrition 99, 559-564, doi:10.1017/50007114507824093 (2007).
12 Hanlon, N., Coldham, N., Gielbert, A. & Sauer, M. Repeated intake of broccoli does not lead to higher plasma levels of sulforaphane in human volunteers. Cancer letters (2009).
13 Ho, E., Clarke, J. D. & Dashwood, R. H. Dietary sulforaphane, a histone deacetylase inhibitor for cancer prevention. The Journal of nutrition 139, 2393-2396, doi:10.3945/jn.109.113332 (2009).
14 Conaway, C., Wang, C., Pittman, B. & Yang, Y. Phenethyl Isothiocyanate and Sulforaphane and their N-Acetylcysteine Conjugates Inhibit Malignant Progression of Lung Adenomas Induced by Tobacco Carcinogens in A/J Mice. Cancer Research (2005).
15 Clarke, J. D., Hsu, A., Yu, Z., Dashwood, R. H. & Ho, E. Differential effects of sulforaphane on histone deacetylases, cell cycle arrest and apoptosis in normal prostate cells versus hyperplastic and cancerous prostate cells. Molecular nutrition &amp; food research 55, 999-1009, doi:10.1002/mnfr.201000547 (2011).
16 Kallifatidis, G. et al. Sulforaphane Increases Drug-mediated Cytotoxicity Toward Cancer Stem-like Cells of Pancreas and Prostate. Molecular therapy: the journal of the American Society of Gene Therapy 19, 188-195, doi:10.1038/mt.2010.216 (2009).
[17] Traka, M. et al. Broccoli consumption interacts with GSTM1 to perturb oncogenic signalling pathways in the prostate. PLoS ONE 3, e2568-e2568, doi:10.1371/journal.pone.0002568 (2008).
18 Dashwood, R. H. & Ho, E. Dietary agents as histone deacetylase inhibitors: sulforaphane and structurally related isothiocyanates. Nutrition reviews 66 Suppl 1, S36-38, doi:10.1111/j.1753-4887.2008.00065.x (2008).
19 Myzak, M., Dashwood, W., Orner, G. & Ho, E. Sulforaphane inhibits histone deacetylase in vivo and suppresses tumorigenesis in Apcmin mice. The FASEB Journal (2006).

What is claimed:

1. A method for controlling, managing, delaying, inhibiting or reversing the proliferation of malignant neoplastic cells, such as seen in disease, but not solely including, such as cancer, which comprises administering an effective amount of a compound or compounds selected from the group consisting of epigallocatechin-3-gallate, curcumin, and a composition comprising glucosinolates and/or derivatives thereof, such as sulforaphane (SFN), and, optionally, providing a modified ketogenic diet or a ketogenic diet to the subject.

2. The composition of claim 1 further comprising medium chain triglycerides (MCT).

3. A method reproducing the key physiological effects of a ketogenic diet of decreasing blood glucose level and increasing blood ketones concentration.

4. A method targeting altered signaling pathways and energy flux in malignant cells.

5. A method enabling horizontal (multiple pathways) and vertical (several steps in a given pathway) targeting of critical pathways supporting tumor robustness and metabolism, cell proliferation and the crosstalk that occurs between the different mechanisms responsible for growth and the development of resistance.

6. The method of claim 1-5 wherein said that one or more components are provided as powder, capsules, tablets, caplets, gel, oil, liquid, emulsion, gel, drink, liquid food product or solid food product.

7. The method of claim 6 wherein component(s) are administered in the form of a unit dosage or separate formulations administered to the subject simultaneously or sequentially.

8. A safe non-toxic intervention comprising the composition of any one of claims 1-7.
